(19) United States
(12) Patent Application Publication
(10) Pub. No.: US 2025/0009691 A1
(43) Pub. Date: Jan. 9, 2025

(54) DIALYSIS FLUID COMPRISING KETONE BODIES FOR THE TREATMENT OF CANCER

(71) Applicant: GAMBRO LUNDIA AB, Lund (SE)

(72) Inventors: Sture HOBRO, Lund (SE); Anders NILSSON, Södra Sandby (SE)

(21) Appl. No.: 18/711,827

(22) PCT Filed: Nov. 29, 2022
(86) PCT No.: PCT/EP2022/083622

§ 371 Date: May 20, 2024

(30) Foreign Application Priority Data
Dec. 1, 2021 (SE) 2151464-1

(51) Int. Cl. A61K 31/19; A61K 31/121; A61K 33/00; A61K 45/06; A61P 35/00

(52) U.S. Cl. CPC A61K 31/19; A61K 31/121; A61K 33/00; A61K 45/06; A61P 35/00

(57) ABSTRACT

The present application provides a dialysis fluid comprising:
a) ketone bodies such as acetoacetate, beta-hydroxybutyrate or pharmaceutically acceptable derivatives, esters and salts thereof; and
b) bicarbonate ions,
   for use in a dialysis therapy method of treating cancer.

The disclosure herein relates to extracorporeal blood treatment. More specifically, the disclosure relates to the use of extracorporeal blood treatments such as hemodialysis, hemodiafiltration, and hemofiltration in the treatment of cancer. Most specifically, the disclosure relates to dialysis fluids for use in hemodialysis therapy treatment methods of cancer.

### TECHNICAL BACKGROUND

Most human cancer cells show an altered energy metabolism that distinguishes them from normal cells. Normal cells acquire most of their energy through mitochondrial oxidative phosphorylation, an aerobic process in which glucose is oxidized, firstly through the glycolysis and subsequently through the tricarboxylic acid (TCA) cycle to produce adenosine triphosphate. This pathway is, on the contrary, only secondary in cancer cells. This was first observed in the 1920s by Warburg. He noted that after cancer cells have metabolized glucose by the glycolysis, lactate is produced from puruvate. In normal cells, this only occurs under anaerobic conditions, but in cancer cells, this alternative pathway is increased even in the presence of abundant oxygen. This phenomenon was named as “aerobic glycolysis” or “Warburg effect” (Warburg et al., 1927, Gen Physiol 8:519-530). The presence of a characteristic glycolytic phenotype in cancer cells was confirmed by subsequent studies that have also observed overexpression of enzymes involved in glycolysis in most of cancer cells.

The above metabolic transformation confers to cancer cells a selective growth advantage and contributes to the ability to resist to hypoxia and apoptosis. Since the rate of tumor cell proliferation exceeds the rate of new blood vessel formation, many tumors grow in a low-oxygen environment. Various metabolic alterations in cancer cells exist and the most common and the most well-known is their habit to produce energy through aerobic glycolysis. Furthermore, many intermediates of glycolysis, such as, for example, ribose, glycerol and serine, are also intermediates of biosynthetic and anabolic pathways that are essential during cancer cell growth and proliferation. Also, glycolysis produces ATP from ADP which allows to sustain cell growth in the tumor. However, glycolysis is much less efficient than oxidative phosphorylation, and therefore requires a high amount of glucose to produce sufficient amounts of ATP. Therefore, this metabolic pathway requires a high amount of glucose. Many cancer cells become addicted to glucose as their main energy supplier. Owing to multiple reasons, glycolytic tumor cells become vulnerable if their glucose supply is targeted.

Further, many cancer cells also display addiction to glutamine. The high rate of glutamine uptake exhibited by glutamine-dependent cells is not only a result from its role as a nitrogen source in nucleotide and amino acid biosynthesis, but also, glutamine is the primary mitochondrial substrate in cancer and is required to produce NADPH for redox control and macromolecular synthesis.

Other metabolic alterations exist in cancers that have an important role in survival and, Importantly, many cancers show a surprisingly good ability to change their metabolic profile. This plasticity to withstand environmental challenges such as when glucose, glutamine, or oxygen get low is crucial for the survival of the cancer cell.

Many metabolic alterations exist in cancers and various amino acids such as glutamine have important roles in cancer metabolism to control redox balance and to produce building blocks for continued proliferation and further, many cancers show a surprisingly good ability to utilize those alternative pathways to change their metabolic profile when needed to adopt to new metabolic limitations. This quality (ability) is an important feature for cancers and their ability to withstand environmental challenges when metabolic energy resources such as glucose, glutamine, or oxygen become scarce.

Therefore, to effectively affect cancer through a metabolic approach it is important to affect their metabolic system from more than one direction. Reduced glucose can by most cancer cells be readily handled, but if several metabolic possibilities are affected at the same time (as reducing glucose and glutamine) the sum of these changes becomes much more devastating than the individual parts.

Beside glucose and glutamine, which are global energy sources for many cancers, serine and glycine meet important specific needs to sustain cell growth and proliferation in cancer, for example through the one-carbon metabolism. In addition to a large energy requirement, cancer cells must also accumulate building blocks for the construction of new cellular components, including nucleic acids, proteins, and lipids, as well as equally important cofactors for the maintenance of their cellular redox status (Amelio et al.: Trends. Biochem. Sci. (2014), vol. 39 (4): 191-198)).

Studies have demonstrated that arginine is necessary for cellular growth and can become limiting in states of rapid growth and if deprived from the cancer cells also effect survival (Albaugh et al., J. Surg. Oncol. (2017), vol. 115 (3), 273-280).

In view of the above, it has been suggested that reduced glycaemia may serve as a strategy to target a broad range of glycolysis dependent tumors. In low glycaemia conditions, fats and especially ketone bodies can replace glucose as a primary metabolic fuel for normal cells. Many tumors, however, have abnormalities in the genes and enzymes needed to metabolize lipids and ketone bodies for energy. Therefore, a transition from carbohydrate to ketones for energy specifically targets the energy metabolism in glycolysis-dependent tumor cells (Seyfried et al, 2010, Nutrition and Metabolism, 7:7).

In accordance with this approach, for example, WO2011070527 discloses a method of treatment of a proliferative disorder, cancerous or non-cancerous, in an individual wherein a hemodialysis apparatus is used to reduce blood glucose concentration.

The use of a hemodialysis apparatus for reducing glycemia has the advantage that the glucose concentration in the blood can be reduced and thereby decreased in a more controlled and effective way compared to diet glucose deprivation. However, the method and apparatus disclosed in WO2011070527 require blood glucose sensors and blood glutamine sensors connected to the blood intake-flow, the blood return flow and the dialysate, which sensors all are connected to the central control unit of the hemodialysis machine. Moreover, the central control unit of WO2011070527 is also connected to an electroencephalograph (EEG) in order to provide the central unit with information pertaining to spontaneous electro-cerebral activity to initiate raising of glucose and glutamine levels. Such a large number of sensors and instruments leads to a high level of complexity and associated high cost. Further, patients undergoing this treatment must consume only a glucose restricted diet for several days prior to undertaking the treatment. This is not an insignificant burden on the patient.

There is a constant need for improved ways of treating cancer. More specifically, there is a need for a dialysis fluid that could be in used hemodialysis therapy treatment methods for treating cancer.

### SUMMARY

The present invention provides a dialysis fluid comprising:
a) ketone bodies such as acetoacetate, beta-hydroxybutyrate or pharmaceutically acceptable derivatives, esters and salts thereof; and
b) bicarbonate ions, for use in a hemodialysis therapy method of treating cancer.

Preferably, the concentration of ketone bodies amounts to 1-15 mM.

More preferably, the concentration of ketone bodies amounts to 2-12 mM.

Preferably, the concentration of bicarbonate ions amounts to 15-50 mM.

More preferably, the concentration of bicarbonate ions amounts to 20-40 mM.

In a preferred embodiment, the sum of the concentrations of glucose, pyruvic acid, and hydrolysis-equivalent concentrations of amino acids selected from the group of serine, cysteine, glycine, alanine, glutamic acid glutamine, proline, aspartic acid, asparagine, threonine, and derivatives thereof, in the dialysis fluid, amounts to at most 3.3 mM

The dialysis fluid may contain one or more compounds selected from the group of glucose, pyruvic acid, and amino acids selected from the group of serine, cysteine, glycine, alanine, glutamic acid, glutamine, proline, aspartic acid, asparagine, threonine or pharmaceutically acceptable derivates thereof. Such derivatives could be salts or oligopeptides. In case the dialysis fluid contains a certain concentration of an oligopeptide, the concentration of the participating amino acids after hydrolysis of the peptide is given (This concentration is referred to herein as “hydrolysis-equivalent concentration”). Examples of such oligopeptides, typically dipeptides where at least one of the amino acid residues is glutamine are L-alanyl-L-glutamine, and L-glycyl-L-glutamine. In case the concentration of the dipeptide L-alanyl-L-glutamine would be 1 mM, the hydrolysis-equivalent concentration of the alanine and glutamine parts, respectively, of this oligopeptide would be 1 mM alanine and 1 mM glutamine. The hydrolysis-equivalent concentration of an amino acid which is not part of an oligopeptide is simply the concentration of the amino acid or amino acid derivative.

Glutamine-containing oligopeptides, also referred to herein as glutamine-containing compounds, are typically used instead of glutamine in liquid compositions in order to enhance stability and solubility.

Preferably, the dialysate fluid does not contain any pyruvic acid, serine, cysteine, glutamic acid, proline, aspartic acid, asparagine, threonine, or derivatives thereof.

Preferably, the dialysis fluid contains a hydrolysis-equivalent concentration of at most 0.3 mM of glutamine or derivatives thereof.

In this disclosure the term “subject” relates to a human or animal patient in need of treatment.

The term “ketone bodies” relates to water-soluble molecules containing the ketone group that may be produced by the liver from fatty acids. Typically, a ketone body in accordance with the present invention is beta-hydroxybuturate or a pharmaceutically acceptable derivative thereof, such as its enantiomer (R)-beta-hydroxybutyric acid, (S)-beta-hydroxybutyrate, or enantiomeric mixture, or a pharmaceutically acceptable salt thereof, or a pharmaceutically acceptable ester thereof, as well as acetoacetate. Medium chain triglycerides are also considered to be derivatives of a ketone body in accordance with the present invention. The term “medium chain triglycerides” or “MCT oils” are triglycerides with two or three fatty acids having an aliphatic tail of 6-12 carbon atoms. Such medium chain triglycerides or MCT oils may be transformed into ketone bodies in the human body. Examples of infusion liquids containing ketone bodies or ketone body derivatives are Lipofundin® MCT/LCT 20% (B. Braun) or SMOFlipid® 20% (Fresenius Kabi). Further examples can be found in WO2018/114309 A1.

Preferably, the cancer is a cancer with metabolic alteration that makes the cancer dependent on glucose and/or glutamine. Typically, the cancer is selected from human colon carcinoma and glioblastoma, as well prostate, breast and liver cancer.

In one embodiment, the dialysis fluid contains a pharmaceutically acceptable amount of a pharmaceutically acceptable cytostatic agent.

The above summary of the present disclosure is not intended to describe each embodiment or every implementation thereof. Advantages, together with a more complete understanding of the present disclosure, will become apparent and appreciated by referring to the following detailed description and claims taken in conjunction with the accompanying drawings.

### BRIEF DESCRIPTION OF THE DRAWINGS

FIG. 1 is a schematic block diagram of an exemplary extracorporeal blood treatment system that can be used for treating cancer by hemodialysis using a dialysis fluid according to the present invention.

FIGS. 2A, 2B and 2C illustrate the treatment goals in accordance with the present invention.

FIGS. 2D and 2E illustrate possible pH shifts in vicinity of normal cells and cancer cells in connection with dialysis using a dialysis fluid according to the present application. FIG. 2F illustrate the hypothesis that peritoneal dialysis using a dialysate fluid according to the present invention renders cancer cells more sensitive to cytostatic agents.

FIGS. 3a, 3b, 3c, 3d, 3e, 3f, 3g, 3h, 3i, 3j and 3k show charts of the results of Study 1.

FIGS. 4a, 4b, 4c and 4d show charts of the results of Study 2.

FIG. 5 shows growth rate of A549 and RCC4 cells in Medium A-C as indicated.

FIG. 6 presents amounts of lung carcinoma A549, renal carcinoma RCC4 and primary RCC cells after 3 days culture in Medium A-C at 21 or 5% oxygen. Stars denotes significantly different values determined by 2way ANOVA with Tukey's multiple comparison test.

FIG. 7 shows results of culture of human glioma cell line A172 at normoxia and hypoxia in Medium A-C with the addition of 8 mM Acac, 16 mM BOHB or the combination of 4 mM Acac/8 mM BOHB. 4 and 8 mM LiCl are used as controls for Acac. Significantly different values determined by one-way ANOVA with Sidak's multiple comparisons test are marked with stars.

FIG. 8 illustrates results of culture of glioma cell line U118MG at normoxia and hypoxia in Medium A-C with the addition of 8 mM Acac, 16 mM BOHB or the combination of 4 mM Acac/8 mM BOHB. 4 and 8 mM LiCl are used as controls for Acac. Significantly different values determined by one-way ANOVA with Sidak's multiple comparisons test are marked with stars.

FIG. 9 presents results of culture of glioma cell line A172 at normoxia and hypoxia in Medium B-C with the addition of 8 mM Acac, 16 mM BOHB or the combination of 4 mM Acac and 8 mM BOHB. 4 and 8 mM LiCl are used as controls for the 4 mM Acac/8 mM BOHB or 8 mM Acac, respectively. Significantly different values determined by one-way ANOVA with Sidak's multiple comparisons test are marked with stars.

FIG. 10 presents results of culture of glioma cell line U118MG at normoxia and hypoxia in Medium B-C with the addition of 8 mM Acac, 16 mM BOHB or the combination of 4 mM Acac and 8 mM BOHB. 4 and 8 mM LiCl are used as controls for the 4 mM Acac/8 mM BOHB or 8 mM Acac, respectively. Significantly different values determined by one-way ANOVA with Sidak's multiple comparisons test are marked with stars.

FIG. 11 presents results of culture of renal carcinoma cell line RCC4 at normoxia and hypoxia in Medium B-C with the addition of 8 mM Acac, 16 mM BOHB or the combination of 4 mM Acac and 8 mM BOHB. 4 and 8 mM LiCl are used as controls for the 4 mM Acac/8 mM BOHB or 8 mM Acac, respectively. Significantly different values determined by one-way ANOVA with Sidak's multiple comparisons test are marked with stars.

FIG. 12 shows a schematic diagram of the experimental setup of Example 3. Hemodialysis was performed in anesthetized ketotic Sprague-Dawley rats using a blood flow rate of 5 ml/min. Blood samples were obtained before and after dialysis, and at 60, 90, 120, 180 min. Samples of the dialysate were taken at 10, 20, 40, 60, 90, 120, 150, and 180 min.

FIGS. 13A, 13B, 13C, and 13D disclose changes in plasma concentration of ketones, glucose, urea and plasma base excess with time during dialysis.

### DETAILED DESCRIPTION OF EXEMPLARY EMBODIMENTS

The present invention relates to a new dialysis fluid for use in a hemodialysis therapy method of treating cancer.

Exemplary systems for extracorporeal blood treatment that could be used together with the dialysis fluids disclosed herein are described in patent application PCT/EP2021/064592. Furthermore, non-limiting examples of systems and devices for carrying out extracorporeal blood treatment can be found in EP1 668 556, U.S. Pat. Nos. 5,679,245, 5,762,805, 5,776,345 and 5,910,252.

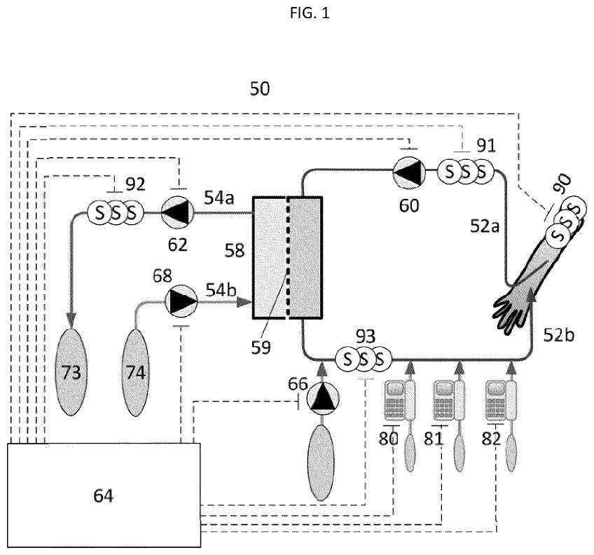

In the schematic view of FIG. 1, the exemplary extracorporeal blood treatment system 50 generally includes a blood tubing circuit 52 which includes an arterial line 52a and a venous line 52b which are connectable to a patient's vascular system. The apparatus comprises a filtration unit 58 having a primary chamber (blood chamber) and a secondary chamber (dialysate chamber) separated by a semi-permeable filtration membrane 59. The inlet of the primary chamber is connected to the blood withdrawal line or arterial line 52a. Likewise, the outlet of the primary chamber is connected to the blood return line or venous line 52b.

The inlet of the secondary chamber is connected to a fresh dialysate fluid supply line 54b, which in turn is connected to a source 74 for providing a fresh dialysis solution. The outlet of the secondary chamber of the filtration unit 58 is connected to a dialysis solution or spent dialysate line 54a, which conveys a spent dialysis solution to an effluent connector 73.

In the present invention, the filtration unit 58 comprises a membrane 59 which differs from conventional hemodialysis filtration membranes in that it is configured to remove relatively small compounds from blood in the blood tubing circuit 112. The membrane is configured to have a molecular weight cut off (MWCO) of 50 kDa or less or even 40 kDa or less (such as 30 kDa or less, 10 kDa or less, 5 kDa or less or 2 kDa or less).

Flow of blood through the blood line 52a, 52b and filtration unit 58 is governed by a blood pump 60 positioned in the arterial line 52a. Likewise, flow of fresh dialysate fluid from the source 74 through the filtration unit 58 to the effluent collector 73 by a dialysate fluid pump 68 in the supply line 54b, while pressure in dialysate fluid line 54 is governed by a pressure release valve in the spent dialysate line 54a. Alternatively, a second pump 62 could be included in the spent dialysate line 54a instead of a pressure release valve.

There could also be one or more infusion lines 66, 80, 81, 82 that are connected to the venous portion of the blood line 52b. In some embodiments, one or more of these infusion lines are adapted to be directly connected to the vascular system of a patient (not shown in the figures). Each of the one or more infusion lines 66, 80, 81, 82 may comprise separate pumps. In one embodiment, an infusion liquid containing ketone bodies or ketone body derivatives could be infused by such an infusion line.

A system computing unit 64 is provided in communication with the blood pump 60, the dialysate fluid pump 68, and the one or more pumps of the one or more infusion lines 66, 80, 81, 82 and the pressure release valve or additional dialysate pump 68 so as to provide control over those devices in use. In an alternative embodiment, the system computing unit 64 provides the user with suitable settings data via a graphical user interface for the pumps in the one or more infusion lines 66, 80, 81, 82. The system computing unit is also in communication with an input means for the provision of information and instruction to the system processing unit 64. The input means may comprise a graphical user interface such as one which may be controlled by the use of a touch screen arrangement. In some embodiments, the input means may comprise a keyboard. In some embodiments, the input means may include one or more sensors for detecting the concentration of various substances or conditions in the patient's blood and/or in the spent dialysate.

As already mentioned, the present invention provides a dialysis fluid for use in a hemodialysis method. The basic idea behind dialysis treatments is to change the composition of a body fluid by using a semipermeable membrane and a dialysate fluid. The term “body fluid” typically relates to blood but may also be an intracellular fluid of a subject to be treated. The body fluid is separated from the dialysate fluid by the membrane. The membrane is permeable for small molecules but impermeable for larger molecules. Small body fluid components may therefore pass the membrane whereas larger entities are retained where they are. The result is that the concentrations of the body fluid as well as the dialysate fluid change. The driving forces of these changes are diffusion and osmotic pressure.

As aerobic glycolysis or the Wartburg effect is common in many cancer cells, the following therapeutic goals may be set up:
a) reduction of the concentration of glucose as well as components of the citric acid cycle and amino acids originating from these components, such as pyruvic acid, serine, cysteine, glycine, alanine, glutamic acid, glutamine, proline, aspartic acid, asparagine, threonine and derivatives thereof, in the body fluid;
b) maintenance of physiological pH (pH within the range of 7.2-7.6) in the body fluid as lactic acid formed during aerobic glycolysis may locally reduce pH; and
c) adding ketone bodies in order to provide a source of energy that tumors dependent on aerobic glycolysis cannot rely opon.

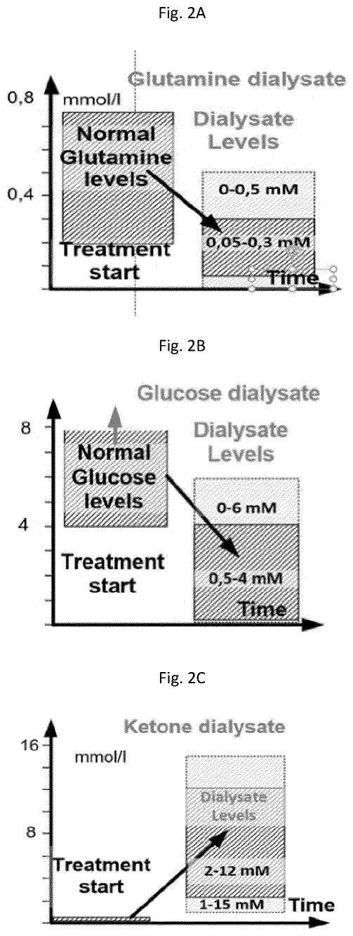

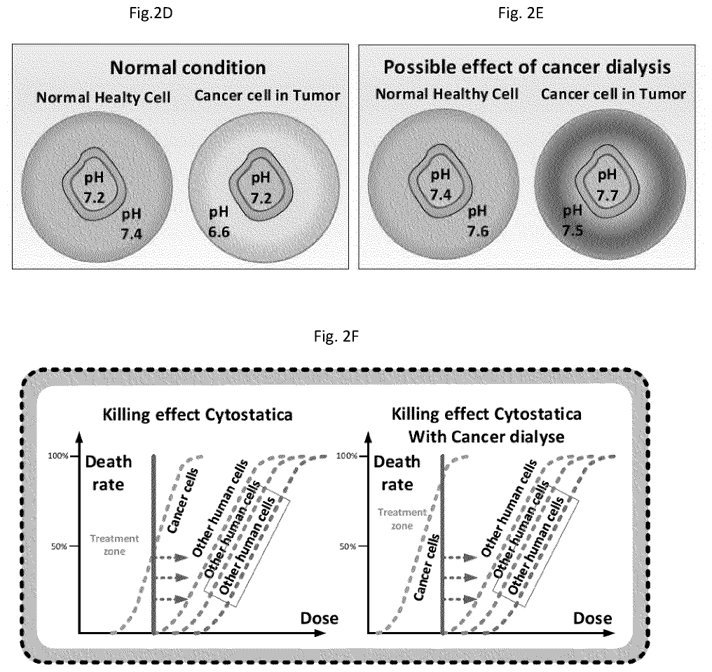

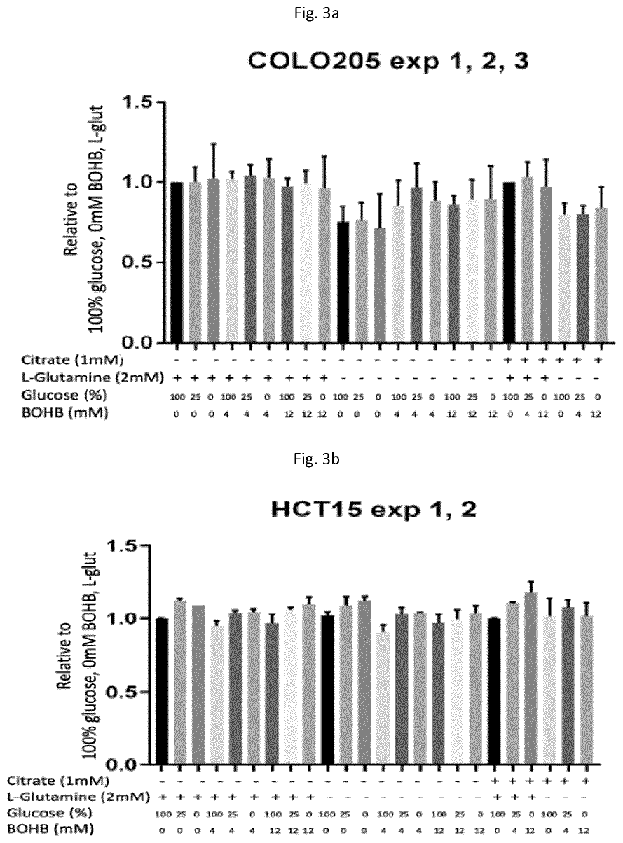

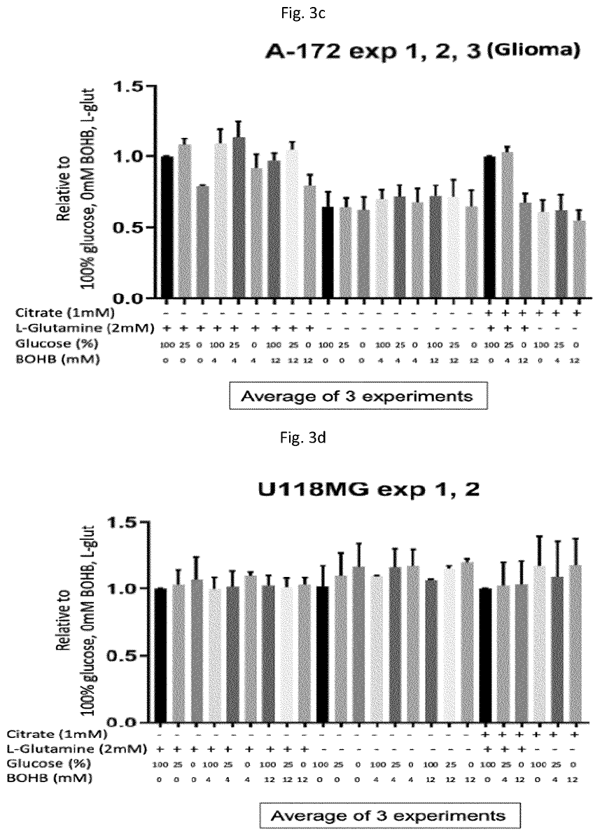

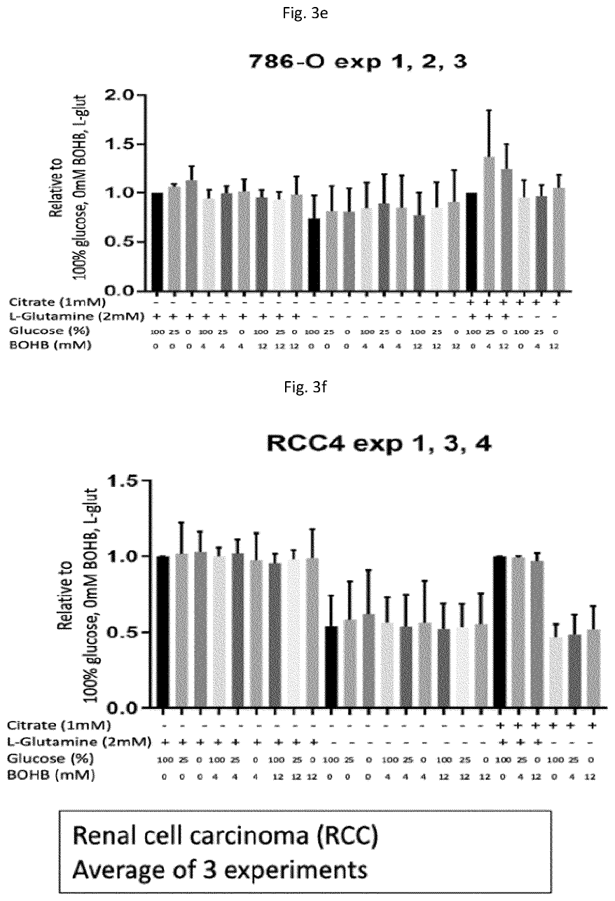

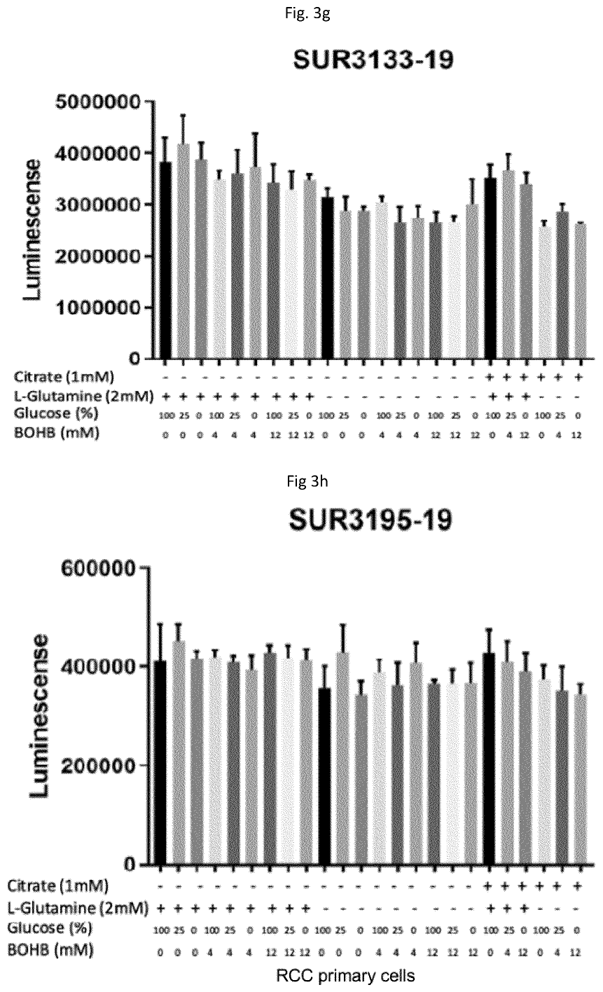

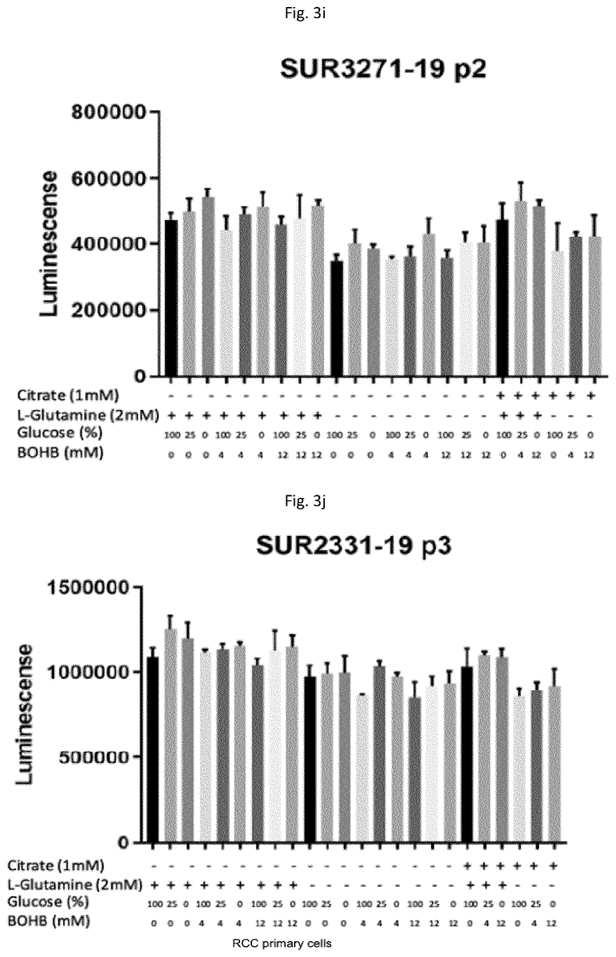

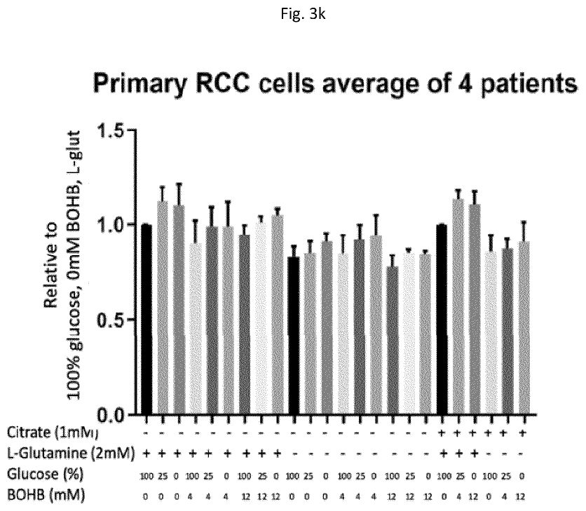

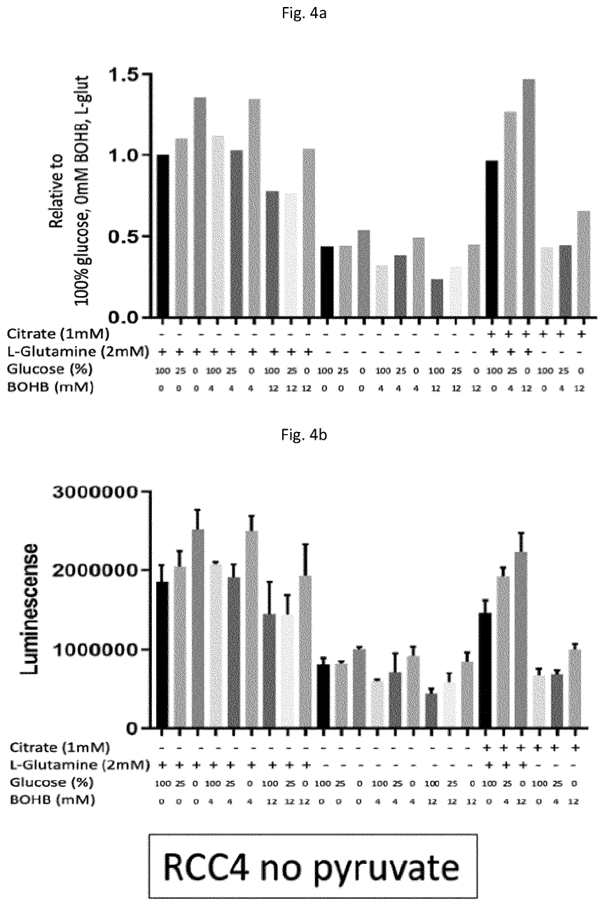

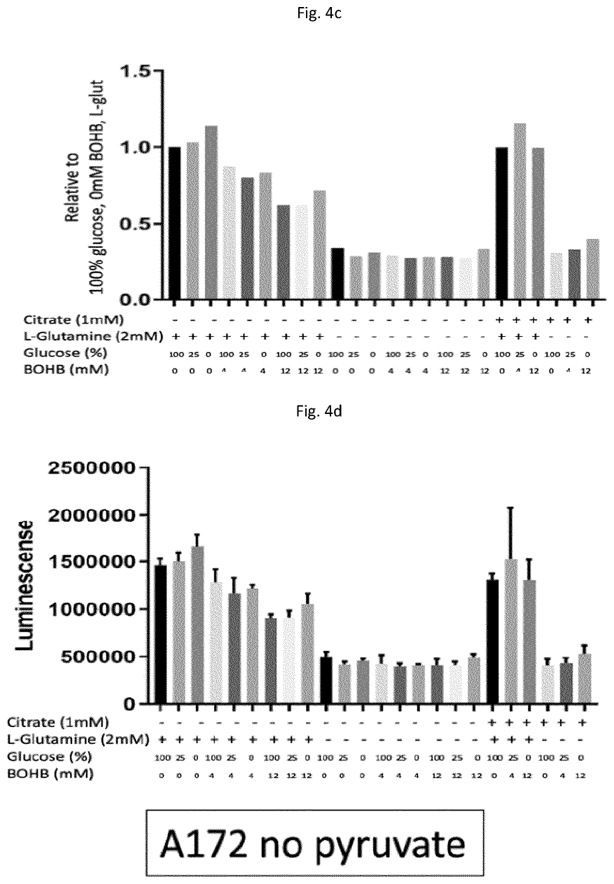

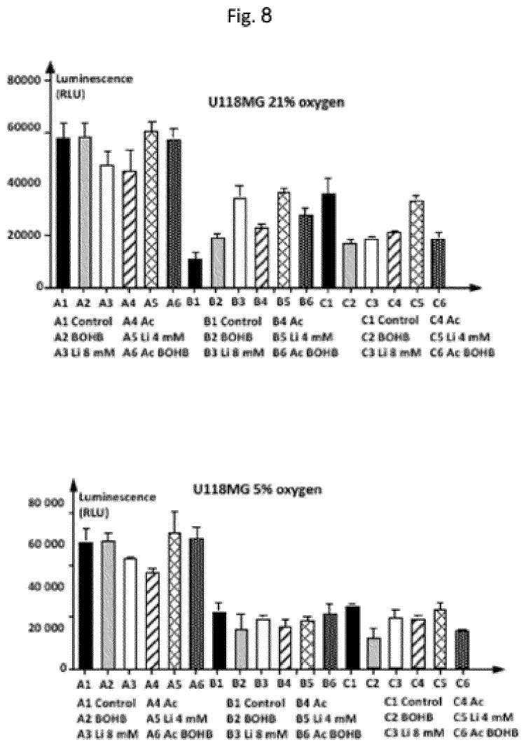

FIGS. 2A, 2B and 2C illustrate examples of some of the therapy goals and principles of the present invention. Each diagram discloses examples of normal conditions regarding concentration of a specific blood component. FIG. 8A shows that the patient at the onset of the treatment typically has an actual blood concentration of glutamine GLNa within the range of 0.20-0.8 mM. During treatment in which a dialysate fluid with no glutamine or a very low glutamine content, the actual blood concentration of glutamine is reduced to a desired value GLN_{b}, which is within the range of 0.1-0.5 mM, and for example within the range of 0.15-0.3 mM. FIG. 2B shows that the patient at the onset of the treatment typically has an actual blood concentration of glucose GLUCOSEₐ within the range of 4-8 mmol/l. During treatment, with a dialysate fluid containing no or a low amount of glucose, the actual blood concentration of glucose is reduced to a desired value GLUCOSE_{b}, which typically is within the range of 2-4 mmol/l. Finally, FIG. 2C shows that the blood initially hardly contains any ketone body such as beta-hydroxy-butyric acid or physiologically acceptable salt or ester thereof, such as the sodium salt. Accordingly, the actual value of concentration of ketone bodies in the patient's blood KETONE, is about 0. Typically, the initially used fresh dialysate does not contain any ketone bodies or only small amounts. During treatment, the blood concentration of such ketones may rise to a desired value KETONE, within the range of 1-15 mmol/l such as within the range of 2-12 mM, by using a second dialysate fluid containing ketone bodies. The dialysate should contain a buffer component such as bicarbonate in order to facilitate maintenance of physiological pH in vicinity of a cancer tumor.

FIG. 2D illustrates pH in the vicinity of normal healthy cells as well as cancer cells under normal conditions. It should be noted that a normal healthy cell is surrounded by a neutral to slightly alkaline pH, whereas a cancer cell, due to production of lactic acid as a consequence of aerobic glycolysis is surrounded by a weak acid pH. Such a pH may have an immunosuppressing effect protecting the cancer cell. Such cancer acidity has been described in Huber et al., Seminars in Cancer Biology, vol. 43 (2017), pp. 74-89.

FIG. 2E shows possible effects of dialysis using the present dialysis fluid on such normal healthy cells and cancer cells. In both cases, the pH values in vicinity of the cells have increased. This increase is caused by two different mechanisms. Firstly, the present dialysis fluid contains no or a very reduced amount of fuel for aerobic glycolysis. Accordingly, less lactic acid id produced. Secondly, the present dialysis fluid also contains buffering and slightly alkaline bicarbonate ions which also increases pH. Bicarbonate is a commonly used buffering substance in dialysis liquids for treatment of end stage renal disease. As a result of the treatment, the cancer cell in FIG. 2E is not surrounded by an acid environment, and therefore not protected by any immunosuppressing layer.

A particular cancer cell in an environment containing low amounts of fuel for aerobic glycolysis having a non-immunosuppressing pH should be weaker than a corresponding cancer cell would be under normal conditions. As dialysis treatment with the present dialysis fluid therefore should weaken such cancer cells, it is postulated that cancer cells treated accordingly should be more sensitive to cytostatic agents. FIG. 2F illustrates this postulate. The two diagrams show death rate for cancer cells as well as different non-cancer cells. As cancer cells grow fast, they are more sensitive to cytostatic agents that typical non-cancer cells. The left diagram outlines the situation without dialysis and the right diagram should show the situation when a dialysis treatment is run using a dialysis fluid according the present invention.

### Reference Example 1

A study was made of the sensitivity of different human cancer cell lines to the presence of glucose, glutamine and ketones in the cell culture medium with concomitant depletion of selected nutrients, to mimic the conditions obtained with cancer dialysis.

Study 1 was performed on a selection of human cancer cell lines established from renal cell carcinoma, colon carcinoma and glioblastoma. In the first study the effect on cell viability of growth in the presence of increasing concentration of the ketone β-hydroxybutyrate, with the concomitant restriction of glucose and glutamine levels. The addition of citrate to the cell culture medium was also tested. Cells were cultured under these conditions for three days, thereafter cell viability was determined. In the first study, the main effect on cell viability was found when glutamine was depleted from the culture medium.

### Materials and Methods

### Cell Culture Conditions

Cell lines established from human colon carcinoma (HCT15, NCI-H508 and COLO205), renal cell carcinoma (769-P, 786-O and RCC4) and glioblastoma (LN-18, A-172 and U-118 MG) were selected for the analysis. All cell lines were obtained from American Type Culture Collection (ATCC, LGC standards, UK) except for RCC4 and HCT15 that was purchased from Sigma-Aldrich (Merck, Germany). In addition, primary human renal cell carcinoma (RCC) cells isolated from patient nephrectomies were included in the study. The culture conditions were as recommended by American Type Culture Collection (ATCC) were followed, that is to grow cells in DMEM medium with the addition of 1 mM sodium pyruvate, which was also added to the RPMI-1640 medium in order to keep the conditions more similar 769-P, RCC4, LN-18, A-172, U-118 MG and the primary RCC cells were cultured in DMEM high glucose medium, while 786-O HCT15, NCI-H508 and COLO205 were cultured in RPMI-1640 medium according to the recommendations from ATCC. To both media 1% penicillin-streptomycin and 10% calf serum was added. Cells were expanded and aliquots frozen according to standard procedures.

Optimal seeding density was determined for each cell line in 96 well plates according to the “Protocol for optimizing cell seeding densities to ensure Log-phase growth”. This Protocol is as follows:
Prepare a single-cell suspension and measure cell counts/viability.
Dilute cells to approx 160,000 cells/mL in complete media. Add 200 μL of cells to the top row of a 96-well plate. Aliquot 100 μL of complete media into all other wells. A small number of media-only control wells are required on each plate to act as a blank.
Repeatedly, dilute the cell preparation 1 part in 2 down the plate using a 12-well channel pipette, i.e., 100 μL cells added to 100 μL media in the row below. Then, add 50 μL of complete media to all wells. Cover the plate.
Incubate the plate overnight at 37° C., 5% CO₂.
Add 50 μL of fresh media to the plate wells to achieve a final volume of 200 UL and incubate for 72 h at 37° C., 5% CO₂.
Measure viability using Cell Titer Glo assay according to manufacturer's protocol.
Plot log cell number against luminescence intensity to identify the concentration of cells at which log growth is achieved.

CellTiter-Glo Luminescent cell viability assay (Promega) was used as a readout of viability.

### Study 1.

For each cell line, optimal number of cells as determined above were seeded into 96-well plates on day 0. The following day, cells were washed in PBS and the media was changed to DMEM (Fisher Scientific) or RPMI-1640 medium (Saveen Werner) without glucose or L-glutamine, with the addition of nutrients as outlined in Table 3 and the file “plate overview”. Three wells were treated for each condition. After 3d incubation in test condition medium, with daily medium changes, cell viability was determined using the CellTiter-Glo viability test. The experiment was repeated three times for each cell line.

TABLE 1
Cancer cell lines and culture medium

| | |
|---|---|
| kidney | |
| 769-P | DMEM high glucose |
| 786-O | RPMI-1640 |
| RCC4 | DMEM high glucose |
| Colon | |
| HCT15 | RPMI-1640 |
| NCI-H508 | RPMI-1640 |
| COLO205 | RPMI-1640 |
| Brain | |
| CRL2610 (LN-18) | DMEM high glucose |
| A-172 | DMEM high glucose |
| U-118MG | DMEM high glucose |

TABLE 2
Normal culture medium content of selected nutrients.

| | | L-glutamine | NaHCO3 | HEPES | NaPyr |
|---|---|---|---|---|---|
| Medium | Glucose(g/L) | (mM) | (g/L) | (mM) | (mM) |
| RPMI-1640 | 2 (11 mM) | 2 | 2 | 25 | 0(1) |
| DMEM high | 4.5 (25 mM) | 4 | 3.7 (1.5) | 0 | 1 |
| glucose | | | | | |

TABLE 3
Test conditions Study 1.

| | | | BOHB (mM) | |
|---|---|---|---|---|
| | Glucose* | 0 | 4 | 12 |
| w glutamine | 100% | +/−citrate | | |
| | 25% | | +/−citrate | |
| | 0% | | | +/−citrate |
| w/o glutamine | 100% | +/−citrate | | |
| | 25% | | +/−citrate | |
| | 0% | | | +/−citrate |

Matrix showing the combination of different growth conditions used in Study 1. *The amount of glucose is presented as the percentage of the concentration present in the standard culture medium for each cell line. Where indicated, 1 mM of citrate was added to the culture medium. The indicated nutrients were added to DMEM (Fisher Scientific) or RPMI-1640 medium (Saveen Werner) without glucose or L-glutamine.

TABLE 4
product order information

| product | company | prod no |
|---|---|---|
| CELL LINES | | |
| 786-O [786-0] (ATCCR CRL-1932 ™) | ATCC | CRL-1932 |
| COLO 205 (ATCC ® CCL-222 ™) | ATCC | CCL-222 |
| A-172 [A172] (ATCCR CRL-1620 ™) | ATCC | CRL-1620 |
| 769-P (ATCC ® CRL-1933 ™) | ATCC | CRL-1933 |
| NCI-H508 [H508] (ATCCRCCL-253 ™) | ATCC | CCL-253 |
| LN-18 (ATCC ® CRL-2610 ™) | ATCC | CRL-2610 |
| U-118 MG (ATCC ®HTB-15 ™) | ATCC | HTB-15 |
| RCC4 plus vector alone Renal cell carcin | sigma | 03112702- |
| | | 1VL |
| HCT-15 HUMAN COLON ADENOCARCINOMA | sigma | 91030712- |
| | | 1VL |
| CELL CULTURE MEDIUM | | |
| DMEM/high glucose with 4.0 mM L-glut, with Sodium | Nordic Biolabs | SH30243.01 |
| Pyruvate | | |
| RPMI 1640 with 25 mM HEPES with L-Glut | Nordic Biolabs | SH30255.01 |
| 500ML DMEM WO GLUC & PHENOL RED | Fisher Sc | 12307263 |
| RPMI Medium 1640, w/o D-Glucose, w/o L- | saveen werner | 01-101-1A |
| Glutamine, 500 ml | | |
| CELL CULTURE PLASTICS | | |
| 96-well plates white, clear bottom, with lid | Fisher Sc | 10517742 |
| | (Corning) | |
| Opaque white tape, BackSeal backing tape | perkin elmer | 6005199 |
| cell strainer 40 um | Saween Werner | 93040 |
| cell strainer 20 um filcons | BD | 340621 |
| VIABILITY ASSAY | | |
| Cell titer Glo | Promega | G7571 |
| CHEMICALS AND ADDITIVES | | |
| DMSO | sigma | D2650-5x5ml |
| Glucose | | |
| L-glutamine | Fisher Sc | 11500626 |
| PEST | Nordic Biolabs | SV30010 |
| trypsin | Nordic Biolabs | SH30042.01 |
| sodium citrate | sigma | PHR1416-1g |
| DL-B-hydroxybutyric acid sodium crystall | sigma | H6501-5g |

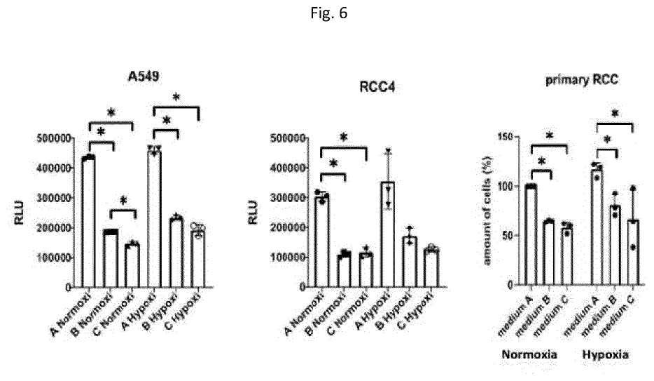

The results of Study 1 are presented in the charts shown in FIG. 6. A clear link is shown between a reduction in glutamine present and the cell culture medium and a reduction in the proliferation of cells.

### Study 2

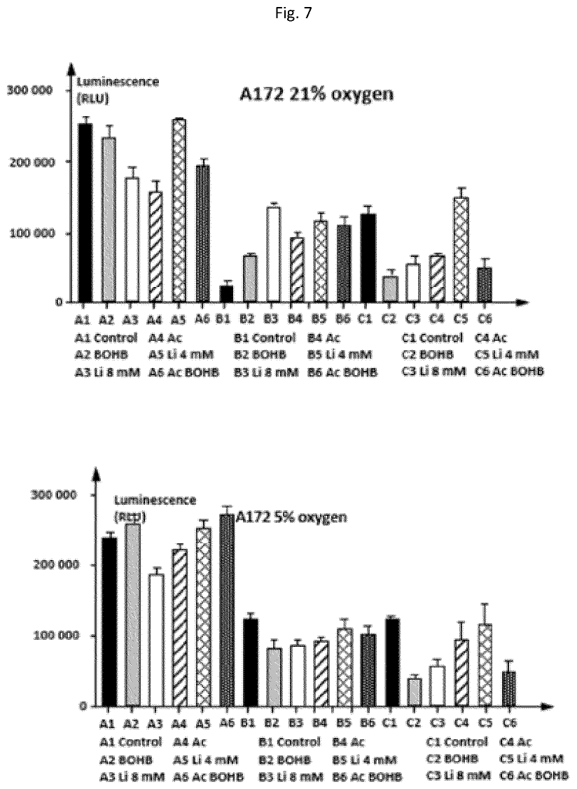

As noted above, in Study 1, the culture conditions recommended from ATCC were followed. However, pyruvate is a potential source of energy that might impact the results. Therefore, in Study 2, the same culture conditions as in Study 1 were tested in two of the cell lines, A172 (glioblastoma) and RCC4 (renal cell carcinoma) in DMEM medium without the addition of sodium pyruvate. The results are presented in FIG. 7. As in Study 1, a clear link is shown between a reduction in glutamine present and the cell culture medium and a reduction in the proliferation of cells. However, in the absence of pyruvate in the cell culture medium, the results are far more pronounced. It also appears that an increase in the concentration of the BOHB ketone also suppresses the proliferation of cells.

All patents, patent documents, and references cited herein are incorporated in their entirety as if each were incorporated separately. This disclosure has been provided with reference to illustrative embodiments and is not meant to be construed in a limiting sense. As described previously, one skilled in the art will recognize that other various illustrative applications may use the techniques as described herein to take advantage of the beneficial characteristics of the apparatus and methods described herein. Various modifications of the illustrative embodiments, as well as additional embodiments of the disclosure, will be apparent upon reference to this description.

### Example 2

### Study Design

### Growth Medium

To study the effect of a nutrient restricted ketogenic environment on cancer cell growth in vitro, three different cell culture media were formulated. Medium A was full RPMI1640 medium that the cell lines routinely are cultured in. Medium B was used as an approximation of the conditions found in normal human serum. The levels of glucose, glutamine, serine, glycine and arginine were adjusted to match normal physiological levels found in human serum. These nutrients were selected based on their reported use as energy source and effects on cancer cell metabolic state. Medium C was used to emulate the ketogenic nutrient restricted Cancer Dialysis condition. Here, the levels of the selected nutrients were reduced to half of the physiological levels in Medium B, and the ketone body BOHB was added.

The composition of each medium is described in Materials and Methods and listed in Table 5-6.

### Oxygen Levels

Human cancer cell lines are routinely established and cultured at atmospheric oxygen levels (21% O₂), yet, the physiological oxygen levels in tissue is considerably lower and varies from 3-13% [Ward, Biochim Biophys Acta 2008; 1777:1-14]. Within the tumor microenvironment, the fast growth rate of cancer cells combined with an often malformed and defective vasculature often results in hypoxic regions with oxygen levels ranging from 0-5%. Given the effects of oxygen levels on energy metabolism [Xie et al., J Biol Chem 2017; 292:16825-16832], and to further mimic the physiological conditions in vivo, the growth of the cancer cell lines in Medium A, B and C was studied at both ambient, 21% O₂, and at the more physiological 5% O₂.

### Ketones

The ketone bodies acetoacetate (Acac), BOHB and acetone are produced by the liver during fasting or starvation. BOHB is the major ketone body in mammals, while Acac constitutes around 20%. The majority of published in vitro studies where the effect of ketones on cancer cells is studied mainly focus on BOHB, however there are studies that suggest different effects from the addition of Acac compared to BOHB [Vallejo et al., J Neurooncol 2020; 147:317-326]. To further mimic the in vivo ketogenic situation where both ketones are present, and to investigate a possible differential effect of BOHB and Acac, Acac was also included in the study.

### Materials and Methods

### Cell Lines

All cell lines were purchased from ATCC (ATCC, LGC standards) except for RCC4 that was from Sigma-Aldrich (Merck). Primary human renal cell carcinoma cells were isolated from nephrectomies performed at Sahlgrenska University Hospital in Gothenburg, Sweden, after informed patient consent and with permit from the regional ethical committee. Optimal seeding density was determined for each cell line in 96-well plates cultured for 3 days in standard cell culture medium.

### Culture Conditions and Additives

Cells were maintained in RPMI-1640 medium (31870-025 GIBCO) with the addition of 10% serum, 200 mM L-glutamine and 1% penicillin-streptomycin (PEST) in humidified chambers at 37° C. and 5% CO₂. For hypoxic conditions (5% O₂), cells were maintained in a Galaxy 14 S CO₂ incubator (Eppendorf) where N₂ was used to adjust the O₂ level to 5%.

Medium A, B, and C were prepared as follows.

Medium A: RPMI1640 (31870-025, GIBCO) with the addition of 1% PEST, 200 mM L-glutamine and 10% dialyzed serum. Dialyzed serum was used to reduce the amounts of small molecules such as amino acids.

Medium B and C were prepared from RPMI1640 modified medium powder without L-glutamine, glucose and amino acids (R9010-01, US Biological Life Sciences). For 1 L medium, 7.4 g powder was dissolved in 900 ml sterile water without heating and 2 g sodium bicarbonate was added. Amino acids listed in Table 5 were added to the same concentration as in full RPMI1640 medium (Table 5). After all additions, the medium was sterilized by filtering through 0.22 um membranes and divided into two bottles.

In Medium B, to mimic physiological conditions, the levels of glutamine, serine, glycine, arginine and glucose were set to the median of measured levels in human serum, based on data from the Mayo Clinic Laboratories (https://www.mayocliniclabs.com/test-catalog/Clinical+and+Interpretive/9265).

To model Cancer Dialysis conditions in Medium C, the levels of these nutrients were reduced to 50% of the physiological levels. As for Medium A, 1% PEST and 10% dialyzed serum were added to Medium B and C. The concentrations of the selected nutrients in Medium A-C are summarized in Table 6. Sodium pyruvate, that is a common addition in cell culture medium, was not present in any of the used media.

TABLE 5
Amino acid concentrations in RPMI1640

| Amino Acids | (g/L) | dissolved in |
|---|---|---|
| L-Asparagine | 0.0568 | H₂O |
| L-Aspartic Acid | 0.02 | 1M HCl |
| L-Cystines | 0.065 | 2M HCl |
| L-Glutamic Acid | 0.02 | 1M HCl |
| L-Histidine | 0.02 | H₂O |
| Hydroxy-L-proline | 0.02 | H₂O |
| L-Isoleucine | 0.05 | 1M HCl |
| L-Leucine | 0.05 | 1M HCl |
| L-Lysine | 0.04 | H₂O |
| L-Methionine | 0.015 | H₂O |
| L-Phenylalanine | 0.015 | 1M HCl |
| L-Proline | 0.02 | H₂O |
| L-Threonine | 0.02 | H₂O |
| L-Tryptophan | 0.005 | 1M HCl |
| L-Tyrosine | 0.024 | 1M HCl |
| L-Valine | 0.02 | H₂O |

TABLE 6
Nutrient composition of Medium A, B and C.

| | | | Medium C |
|---|---|---|---|
| | Medium A | Medium B | cancer |
| | RPMI-1640 | physiological | dialysis |
| Glucose (mM) | 11 | 5.85 | 2.93 |
| BOHB (mM) | 0 | 0 | 8 |
| Glutamine (uM) | 2000 | 664 | 332 |
| Serine (uM) | 280 | 125 | 62.5 |
| Glycine (uM) | 130 | 308 | 154 |
| Arginine (uM) | 1150 | 76 | 38 |

Amino acids and other additives were purchased from Sigma Aldrich.

After all nutrients were added pH was measured. The pH values were as follows: Full RPMI1640 with 10% non-dialyzed FBS, 1% PEST and 200 mM L-glutamine, pH 7.78; Medium A, pH 7.56; Medium B, PH 7.62 and Medium C, PH 7.57.

Stock solutions of DL-β-Hydroxybutyric acid sodium salt (H6501, Sigma Aldrich) and Lithium Acetoacetate (A8509, Sigma Aldrich) were prepared in water, sterile filtered, aliquoted and stored at −20° C. Lithium Chloride (L7026, Sigma Aldrich) was used as a control for the addition of Lithium in the Li-Acac.

### Viability Assay

CellTiter-Glo Luminescent cell viability assay (Promega) was used as a readout of cell numbers according to the manufacturers' instructions. In experiments shown in FIGS. 7-8, double plates were seeded and treated. One set of plates was used for collection of medium for lactate measurement (see below) and the CellTiterGlo-assay. The other set of plates was frozen at −80° C. at the end of the experiment. The frozen plates were intended for the CyQuant cell proliferation assay (Thermo Fisher) that measures the amount of DNA per well. Analysis of the amounts of cells by both CellTiterGlo and CyQuant assays would ensure that effects of the culture conditions on viability or growth rate would not be concealed by simultaneous changes in ATP-levels per cell.

### Collection of Medium for Lactate Measurement

In experiments shown in FIGS. 7-8, cell culture medium was collected on day 3, transferred to new 96-well plates and frozen at −80° C. This medium could be used to analyze the amount of excreted lactate as a measurement of metabolic state. Several kits for lactate measurement are available, for example the Lactate-Glo assay (J5021, Promega) is designed for use in assays where serum is present.

### Results

Example 2 was designed to answer the following question:
Is the growth of the selected cancer cell lines affected by the Cancer Dialysis conditions emulated in Medium C at normoxia or hypoxia?

### Growth in Medium a, B and C

As a first step, Medium A-C were prepared as described in Materials and methods, and the ability of the cancer cell lines to grow in these media were tested. Growth curves for the selected cell lines over time in each medium were established. The number of cells was analyzed after 1, 2 and 3 days of culture in medium A-C at normoxia (21% O₂).

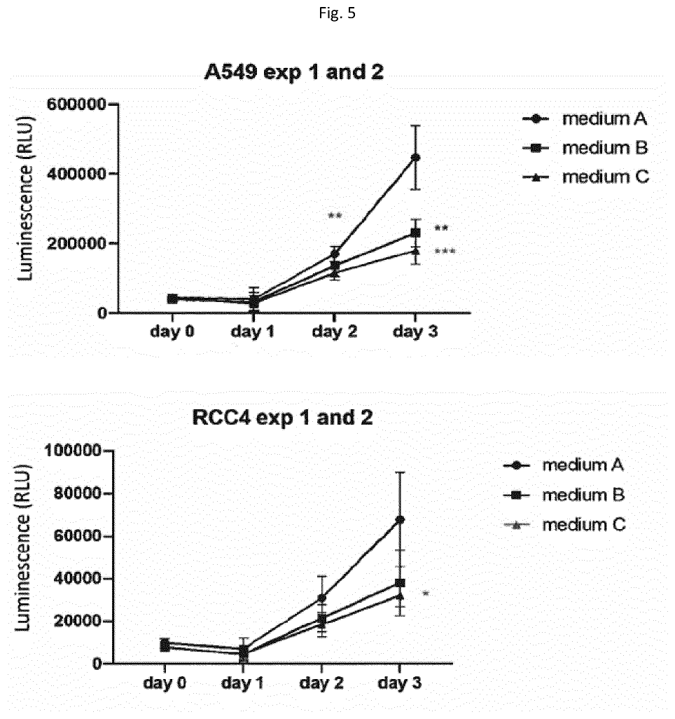

As shown in FIG. 5, reduction of the selected nutrients to more physiological levels as in Medium B reduced the growth rate of A549 lung carcinoma and RCC4 renal carcinoma cell lines significantly compared to Medium A. Medium C further reduced growth rates compared to Medium A.

### Growth of Cancer Cells in Medium A-C at Normoxia and Hypoxia

In FIG. 6, the amounts of cells after 3 days of culture in Medium A-C in normoxia and hypoxia are shown for the A549 lung carcinoma and RCC4 renal carcinoma cell lines as well as for primary renal carcinoma (RCC) cells. Again, the growth rate at normoxia was reduced in Medium B and C compared to Medium A. The same pattern was found in cells cultured at 5% O₂. Again, in RCC4, the low nutrient levels and addition of BOHB in Medium C did not have a significant additional effect compared to the conditions in Medium B.

For A549, a small but significant reduction in growth rate was found between Medium B and C, but only at normoxia.

Primary renal carcinoma cells from three patients were included in this study. Similar to the established cell lines, these cells showed a reduced growth rate in Medium B and C compared to Medium A.

Overall, changing the oxygen pressure from 21% (normoxia) to 5% O₂ (hypoxia) had very limited effect on the growth rate of these cells.

Next, it was decided to also include Acac in the study and analyze the viability of cells cultured in the presence of BOHB and Acac alone or in combination in Medium A-C. The experiment was performed at 21% and 5% O₂. The experiments were made with glioma cell lines A172 and U118MG.

Being a chiral molecule, BOHB exists as two enantiomers, D- and L-BOHB. D-BOHB is normally produced and metabolized in humans. The BOHB-salt used in this study contains a mixture of 50:50 of D- and L-BOHB. In order to ensure a high presence of the active D-form, the total concentration of BOHB added was increased to 16 mM, giving a level of 8 mM D-BOHB. To keep the total concentration of active ketones constant, 8 mM Acac was used, and for the combination of both ketones, the levels were adjusted to 4 mM Acac and 8 mM BOHB (containing 4 mM D-BOHB).

The Acac available for in vitro use is in the form of a lithium salt. Since lithium itself can affect the viability of cancer cells [Cohen-Harazi et al., Anticancer Res 2020; 40:3831-3837], 8 mM LiCl was used as a control for the 8 mM Acac data point and 4 mM LiCl was used as a control for the 4 mM Acac/8 mM BOHB data.

At the end of the experiment, the culture media from each well was collected and frozen to enable later determination of lactate levels as a measurement of the metabolic state. In addition, double experiments were performed, where one set of plates were frozen for later quantification of cell numbers using the CyQuant proliferation assay, and the amounts of cells in the other set were analyzed by CellTiterGlo viability assay.

### The Effect of BOHB and Acac Added Alone or in Combination

As shown in FIGS. 7-8, the initial experiment gave promising data regarding the effect of high ketone concentration in nutrient reduced conditions on the growth of A172 and U118MG glioma cell lines.

In Medium A, at both normoxia and hypoxia, addition of 16 mM BOHB alone had no growth inhibitory effect in A172 or U118MG cells, and 8 mM Acac did not reduce the number of cells further compared to the 8 mM LiCl control.

However, addition of 4 mM Acac in combination with 8 mM BOHB significantly reduced the number of A172 cells in Medium A at normoxia compared to the 4 mM LiCl control.

The interpretation of the results from Medium B was disturbed by a technical error in the normoxic control sample. However, in hypoxic A172 cells, BOHB significantly reduced the number of cells to approximately 70% of the amount in Medium B without BOHB. A similar reduction was also seen in hypoxic U118MG cells.

Furthermore, in Medium B, 8 mM Acac reduced the number of cells significantly compared to the 8 mM LiCl control in both cell lines, but only at 21% O₂.

In Medium C, addition of 8 mM Acac alone did not significantly reduce the amounts of cells compared to the 8 mM LiCl control. However, in both cell lines and at both 21 and 5% O₂, addition of 16 mM BOHB significantly reduced the number of cells to around 30% compared to C-medium without BOHB.

Also the combination of BOHB and Acac resulted in significantly fewer cells in Medium C compared to the 4 mM LiCl control, in both cell lines and at both oxygen levels.

These results suggest that addition of high levels of BOHB or Acac alone do not inhibit the growth of glioma cells in a nutrient rich environment such as Medium A. Also in Medium B, with more physiological nutrient levels, only small differences were seen when the ketones were added. The largest effects were found in Medium C, where both 16 mM BOHB alone and the combination of 8 mM BOHB with 4 mM Acac significantly reduced the number of cells compared to their respective controls, at both hypoxia and normoxia. This was not seen when 8 mM Acac was added alone.

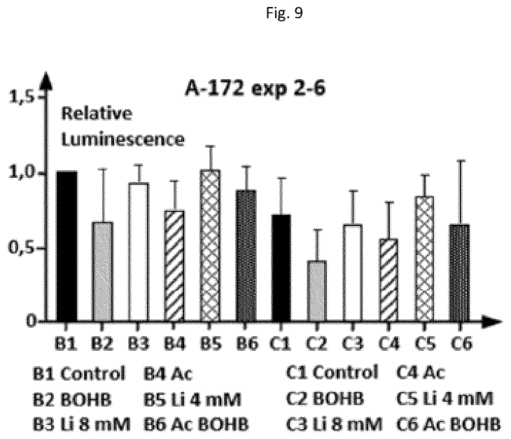

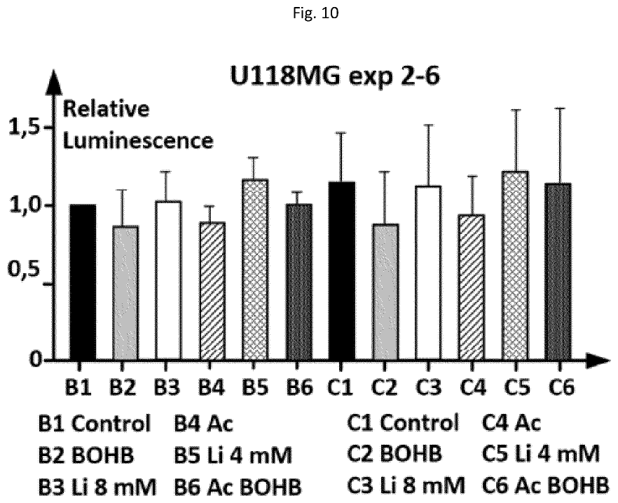

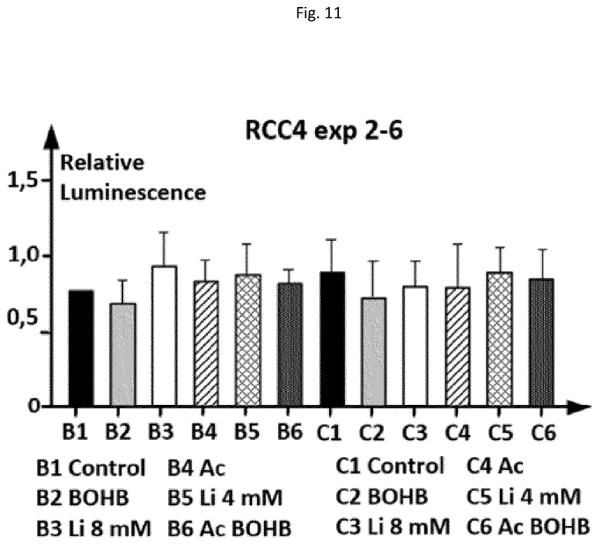

However, repetition of these experiments, with focus on Medium B and C at normoxic conditions, gave inconsistent results. FIGS. 9-11 show the combined results from experiment 2-6, for the glioma cell lines A172 and U118MG and the renal carcinoma cell line RCC4. A trend of reduced growth was seen in Medium C when BOHB or Acac were added separately, especially in the A172 cell line.

The results from the first and second parts of Example 2 indicated that the cancer cell lines grew slower in a nutrient restricted environment. The tested cell lines seemed viable in Medium B and C although the proliferation rate was reduced. Optical inspection of the cells at day 3 did not reveal any floating cells, which could have been a sign of dead cells.

The data from the third part of the study shows an increased sensitivity of glioma cell lines to high levels of BOHB or a combination of BOHB and Acac in the nutrient restricted Medium C. Such a sensitivity was, however, not found for the renal carcinoma cell line RCC4.

### Example 3

In this example, rats were given water and a ketogenic diet followed by dialysis. The effects of dialysis on blood ketones, lactate and actual HCO₃ were determined.

### Methods

### Animals

Experiments were performed in six male Sprague-Dawley rats having an average body weight of 316 g (305-318) given water and ketogenic food (Kliba-Nafag 2201 Ketogenic diet XL75: XP10) five days prior to experiments. The animals were treated according to the guidelines of the National Institutes of Health for Care and Use of Laboratory animals. The Ethics Committee for Animal Research at Lund University approved of the experiments (Dnr 5.8.18-08386/2022). The rat was carefully placed into a covered glass container to which a continuous supply of 5% isoflurane in air (Isoban, Abbot Stockholm, Sweden) was connected. After fully anesthetized, the rat was gently lifted from the container. Anesthesia was maintained using 1.6-1.8% isoflurane in air delivered in a small mask. Following tracheostomy, the rat was connected to a volume-controlled ventilator (Ugo Basile; Biological Research Apparatus, Comerio, Italy) using a positive end-expiratory pressure of 4 cm H₂O. Body temperature was kept between 37.1° C. to 37.3° C. controlled using a feedback-controlled heating pad. End-tidal pCO₂ was maintained between 4.8 and 5.5 kPa (Capstar-100, CWE, Ardmore, Pa). The right femoral artery was cannulated for continuous monitoring of heart rate and mean arterial pressure (MAP); and for obtaining blood samples (95 μL) for measurement of glucose, urea, electrolytes, hemoglobin, and hematocrit (I-STAT, Abbott, Abbott Park, IL), and blood ketones (FreeStyle Precision Neo, Abbott, Abbott Park, IL). The right femoral vein was cannulated and connected to the dialyzer using plastic tubing. The right femoral artery was also cannulated and connected both to a pressure transducer to continuously monitor arterial line pressure, and to a blood pump (Masterflex Ismatec, Cole-Parmer, IL) connected to the dialyzer via plastic tubing. Before connection, the blood circuit was primed with 4% albumin (Albunorm, Octapharma Nordic AB, Sweden) to which heparin 50 IE (Heparin LEO 5000 IE/ml, Leo Pharma AB, Sweden) had been added. The dialysis circuit was connected to a pump at the inlet from a glass cylinder containing fresh dialysis fluid (Hemosol BO, Baxter Healthcare, IL), having the following composition:

| Active substances | mmol/L |
|---|---|
| Sodium Na⁺ | 140 |
| Calcium Ca2⁺ | 1.75 |
| Magnesium Mg2⁺ | 0.5 |
| Chloride Cl⁻ | 109.5 |
| Hydrogen carbonate HCO₃⁻ | 32 |
| Lactate | 3 |

The outlet from the dialyzer was also connected to a peristaltic pump which pumped spent dialysate to a glass cylinder. Both glass cylinders were placed on a scale to ensure that no fluid was removed from the animal (FIG. 12). The right internal jugular vein was cannulated for infusion of maintenance fluid containing ⁵¹Cr-EDTA. Hematocrit was determined by centrifugating thin capillary glass tubes. After the experiment, animals were euthanized with an intravenous bolus injection of potassium chloride.

### Experimental Protocol

A three-hour hemodialysis session was performed using a mini-capillary dialyzer device obtained from Baxter Healthcare (Hechingen, Germany), comprising a high-flux membrane (Polyflux Revaclear®, HF-Revaclear®). Arterial blood samples were obtained before and 30 min after dialysis, and at 60, 90, 120, and 180 min during HD. Dialysate samples were collected before dialysis and at 10, 20, 40, 60, 90, 120, 150 and 180 min and analyzed on a gamma counter (Wizard 1480, LKP Wallac, Turku, Finland) to determine ⁵¹Cr-EDTA activity.

### Statistical Methods

Data are shown as median (interquartile range) unless otherwise specified. Significant differences were assessed using an asymptotic Friedman omnibus test (coin package) which, if significant, was followed by a Wilcoxon-Nemenyi-McDonald-Thompson post hoc test. Two blood ketone values were above the range of measurement (8.0 mmol/L). Before statistical analysis they were set to 8.0 mmol/L. Since they were the highest values in the dataset, a value of 8 mmol/L ensures that (i) the median (IQR) is unaffected, and (ii) that the datapoints will get the highest and the same rank. P-values below 5% were considered significant. Calculations were performed using R for mac version 4.1.1.

### Results

### Baseline Parameters

Six Sprague-Dawley rats were given ketogenic diet for five days and gained between (min-max) 10 to 37 g in body weight. Before dialysis, median arterial pH was 7.43 (7.42 to 7.44), actual bicarbonate was 20.4 mmol/L (19.8 to 21.3), standard base excess was-3.5 mmol/L (−4.0 to −2.2), pO₂ was 12.4 kPa (12.2 to 12.7) and pCO₂ was 4.0 kPa (4.0 to 4.3). Dialysis was performed for three hours after which blood was returned to the animal, followed by a 30 min rest period before termination of the experiment. Treatment and baseline parameters were as follows:

| Parameter | Value | |
|---|---|---|
| Baseline parameters | | |
| Mean arterial pressure, mm Hg | 69 | (62-73) |
| Heart rate | 329 | (315-356) |
| Arterial oxygen saturation (SO₂) % | 98 | (97-98) |
| Plasma sodium, mmol/l | 138.5 | (138.0-139.8) |
| Plasma potassium, mmol/l | 3.8 | (3.8-3.9) |
| Plasma chloride, mmol/l | 103 | (103-103) |
| Plasma ionized calcium, mmol/l | 1.28 | (1.27-1.34) |
| Plasma lactate, mM | 2.9 | (2.1-3.9) |

| | |
|---|---|
| Treatment parameters | |
| Blood flow (QB), mi/min | 1.0 |
| Dialysate flow (Qd), ml/min | 5 ml/min |
| Dialysate solium, mM | 140 |
| Dialysate potassium, mM | 4 |
| Dialysate bicarbonate, mM | 32 |
| Dialysate calcium, mM | 1.75 |
| Dialysate lactate, mM | 3 |

Hemodialysis does not Significantly Reduce Blood Ketone Levels

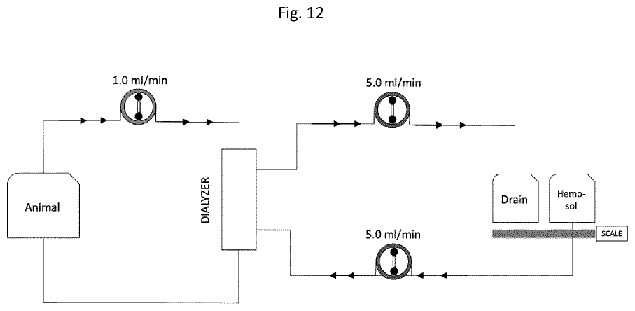

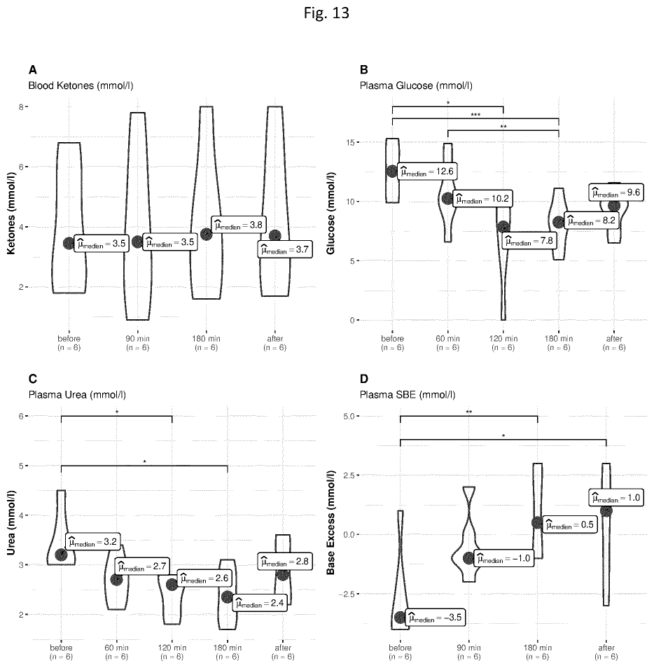

Blood ketone levels before and after 180 min dialysis were similar, being 3.5 mmol/l (2.2-5.6) and 3.8 mmol/l (2.2-5.1), respectively (P=0.53), with no significant differences in ketone concentrations during dialysis (FIG. 13A). The data is shown in the following table:

| Group | Ketones mmol/l | Lactate mmol/l | Actual HCO₃⁻ mmol/l |
|---|---|---|---|
| Before dialysis | 3.5 (2.2-5.6) | 2.9 (2.0-3.9) | 20.4 (19.8-21.3) |
| 90 min | 3.5 (2.0-5.5) | 1.9 (1.3-2.8) | 22.8 (22.4-24.2) |
| 180 min | 3.8 (2.2-5.1) | 2.0 (1.9-2.2) | 23.5 (22.1-24.9) |
| 30 min after dialysis | 3.7 (2.2-5.6) | 1.6 (1.5-1.8) | 23.5 (22.1-25.0) |
| Asymptotic | | | |
| Friedman Test | | | |
| P-value | 0.49 | 0.39 | 0.02 |
| 95% CI for dialysis | [−0.2 to 0.4] | [−0.3 to 0.5] | [0.3 to 0.6] |
| effect | | | |

Urea reduction ratio (URR) was 38% (25-42) and glucose reduction ratio was 36% (29-43), with concentrations decreasing over the course of the treatment (FIG. 13C, 13B). The data is shown in the table below:

| | Sodium | Potassium | Chloride | Calcium | Glucose |
|---|---|---|---|---|---|
| Group | mmol/l | mmol/l | mmol/l | mmol/l | mmol/l |
| Before | 139 | 3.8 | 103 | 1.28 | 12.6 |
| dialysis | (138-140) | (3.8-3.9) | (103-103) | (1.27-1.34) | (10.4-14.5) |
| 60 min | 138 | 4.2 | 103 | 1.32 | 10.2 |
| | (138-139) | (4.1-4.3) | (102-104) | (1.31-1.34) | (9.8-12.2) |
| 120 min | 138 | 4.7 | 103 | 1.33 | 7.8 |
| | (137-138) | (4.5-4.7) | (101-104) | (1.30-1.36) | (5.6-9.4) |
| 180 min | 138 | 4.5 | 103 | 1.27 | 8.2 |
| | (137-138) | (4.3-4.7) | (101-104) | (1.25-1.30) | (6.9-9.1) |
| 30 min | 138 | 4.2 | 102 | 1.22 | 9.6 |
| after | (137-138) | (4.0-4.7) | (101-103) | (1.21-1.26) | (8.2-9.9) |
| dialysis | | | | | |
| Asymptotic | | | | | |
| Friedman | | | | | |
| Test | | | | | |
| P-value | 0.23 | 0.01 | 0.11 | 0.004 | 0.0002 |
| 95% CI for | [−0.3 to 0.4] | [0.2 to 0.7] | [−0.1 to 0.5] | [0.37 to 0.75] | [−0.9 to −1.0] |
| dialysis | | | | | |
| effect | | | | | |

The blood to dialysate clearance of ⁵¹Cr-EDTA was stable during the entire dialysis session being 1.0 ml/min (0.7-1.1). Single-pool Kt/V urea was 0.57 (0.52-0.63), calculated as follows Kt/V=−log(1-URR-0.024). Assuming a total body water of 70% of body weight, this implies a urea plasma to dialysate clearance of 0.67 ml/min (0.62 to 0.80).

### Effects on Acid-Base and Blood Chemistry During Dialysis

Plasma base excess increased during dialysis (FIG. 13D). In line with this, arterial pH increased from 7.42 (7.42-7.44) to 7.51 (7.49-7.52) after 180 min dialysis (P=0.005). Arterial pCO₂ was not significantly different after 180 min hemodialysis, being 4.0 (4.0-4.3) kPa before, and 4.0 (3.7-4.2) kPa after dialysis, respectively (P=0.81). Blood hemoglobin levels were not significantly different before versus after dialysis, being 113 g/L (106-118) and 116 g/L (111-118), respectively (P=1.00). Plasma potassium increased from 3.8 mmol/l (3.8-3.9) before dialysis to 4.5 mmol/L after 180 min (P=0.03), whereas there was no significant difference in plasma sodium (Table 3).

### Discussion

The present example shows that blood ketone levels in rats fed with ketogenic diet were relatively unaffected by hemodialysis, despite the fact that the low molecular weight of ketones (β-hydroxybutyrate, acetoacetate and acetone) means that they will be removed efficiently by dialysis. Indeed, molecules of the same approximate size as keto-acids were effectively removed (e.g. urea), and even bigger molecules like ⁵¹Cr-EDTA were effectively cleared from blood.

The dialysate blood flow rate (Qb) was here set to 1 ml/min, which is similar to other experimental models of hemodialysis in rats (Fukunaga et al., PLOS One. 2020; 15:e0233925). Pre-filter arterial pressures were stable and positive at this blood flow rate, with 40-50 mmHg being a typical value. In order to elucidate how this blood flow rate would correspond to a human undergoing hemodialysis it may be set in relation to the distribution volume. For example, for urea the distribution volume is approximately equal to total body water (TBW), being about 70% of body weight in rats. Thus, a 300 g rat has a TBW of 210 ml, meaning that a blood flow rate of 1 ml clears ˜0.47% of TBW from urea. For a 70 kg adult having a TBW of 42 L this corresponds to a blood flow of 42×0.47%=200 ml/min. Nowadays, blood flow rates are usually 250 ml/min or higher, especially in patients treated with hemodiafiltration. On the other hand, we used a dialysate flow rate of 5 ml/min which is far higher than Qb meaning that solute transport will be limited by the blood flow rather than the dialyzer membrane or dialysate flow. Indeed, in line with this, the estimated plasma to dialysate clearance of urea and ⁵¹Cr-EDTA (a much larger molecule than urea) was nearly identical being 0.67 ml/min for urea and 0.66 ml/min for ⁵¹Cr-EDTA.

We also found that ketogenic diet leads to mild metabolic acidosis in rats, also there was a very mild increase in plasma lactate. A slightly elevated lactate has previously been observed in cows fed with ketogenic diet (Zhang et al., Research in Veterinary Science, 2016, 107:246-256). Also, potassium levels were slightly increased during dialysis to 4.5 mmol/l. This may be due to the fact that potassium 20 mmol was added to a 5 L bag of fresh dialysate manually, which due to tolerances in volume and composition of the potassium solution may cause the actual dialysate concentration to differ from 4 mmol/l. Lastly, there was a slight hyperglycemia before dialysis which is in line with previous studies in mice (Meidenbauer et al., Faseb Journal, 2013, 27) in which they also noted lower insulin levels following ketogenic diet.

1. A dialysis fluid comprising:
a) one or more ketone bodies selected from the group consisting of acetoacetate, beta-hydroxybutyrate or pharmaceutically acceptable derivatives, esters and salts thereof; and
b) bicarbonate ions,
wherein the dialysis fluid is for use in a hemodialysis therapy method of treating cancer.

2. The dialysis fluid according to claim 1, wherein a concentration of one or more ketone bodies amounts to 1-15 mM.

3. The dialysis fluid according to claim 2, wherein the concentration of one or more ketone bodies amounts to 2-12 mM.

4. The dialysis fluid according to claim 1, wherein a concentration of bicarbonate ions amounts to 15-50 mM.

5. The dialysis fluid according to claim 4, wherein the concentration of bicarbonate ions amounts to 20-40 mM.

6. The dialysis fluid according to claim 1, wherein a sum of a concentration of glucose, a concentration of pyruvic acid, and hydrolysis-equivalent concentrations of amino acids selected from the group of serine, cysteine, glycine, alanine, glutamic acid, glutamine, proline, aspartic acid, asparagine, threonine and derivatives thereof, amounts to at most 3.3 mM.

7. The dialysis fluid according to claim 6, wherein the dialysis fluid does not contain any pyruvic acid, serine, cysteine, glutamic acid, proline aspartic acid, asparagine, threonine or derivatives thereof.

8. The dialysis fluid according to claim 7, wherein the dialysis fluid comprises the hydrolysis-equivalent concentrations of amino acids of at most 0.3 mM glutamine or derivatives thereof.

9. A dialysis fluid according to claim 1, wherein the dialysis fluid comprises a pharmaceutically acceptable amount of a pharmaceutically acceptable cytostatic agent.
